(19) United States
(12) Patent Application Publication
(10) Pub. No.: US 2002/0037854 A1
(43) Pub. Date: Mar. 28, 2002

(54) USE OF AT LEAST ONE NO SYNTHASE INHIBITOR FOR TREATING SENSITIVE SKIN

(76) Inventors: LIONEL BRETON, VERSAILLES (FR); OLIVIER DE LACHARRIERE, PARIS (FR)

Correspondence Address: BURNS DOANE SWECKER & MATHIS L L P, POST OFFICE BOX 1404, ALEXANDRIA, VA, 22313-1404, US

(21) Appl. No.: 08/981,992

(22) PCT Filed: Oct. 1, 1996
(86) PCT No.: PCT/FR96/01529

(30) Foreign Application Priority Data
Oct. 26, 1995 (FR) 95/12653

(51) Int. Cl. A61K 38/00; C07C 241/00; C07C 243/00; C07C 249/00; C07C 251/00; C07C 259/00; C07C 273/00; C07C 275/00; C07C 277/00; C07C 279/00; C07C 291/00; A61K 31/195; A01N 37/12; A01N 37/44

(52) U.S. Cl. 514/18; 514/19; 562/560

(57) ABSTRACT

The invention is directed to a method for preventing or treating the symptoms associated with sensitive skin by applying an effective amount of at least one nitric oxide synthase inhibitor to treat or prevent the symptoms associated with sensitive skin to an individual in need thereof.

The present invention relates to the use of at least one NO synthase inhibitor in a cosmetic composition as active principal for treating and/or preventing sensitive skins, or for the preparation of a pharmaceutical composition, more particularly a dermatological composition, intended for treating sensitive skins.

In the field of cutaneous disorders, it is known that certain skins are more sensitive than others. Nevertheless, the symptoms of sensitive skins were poorly characterized until now and the problem of these skins was therefore poorly defined; nobody knew exactly the process implicated in the sensitivity of the skin. Certain people thought that a sensitive skin was a skin which reacted to cosmetic products, others that it was a skin which reacted to several external factors, not inevitably linked to cosmetic products. Sensitive skins were likewise compared to allergic skins.

Tests have been perfected for defining sensitive skins, for example tests with lactic acid and with DMSO which are known to be irritant substances: see, for example, the article of K. Laimintausta et al., Dermatoses, 1988, 36, pp. 45-49; and the article of T. Agner and J. Serup, Clinical and Experimental Dermatology, 1989, 14, pp. 214-217.

As a result of the lack of knowledge of the characteristics of sensitive skins, it was until now very difficult or even impossible to treat them. In fact, they were treated indirectly, for example by limiting the use of products of irritant character such as surfactants, preservatives or perfumes as well as the use of certain cosmetic or dermatological active compounds in cosmetic or dermatological compositions.

After numerous clinical tests, the Applicant has been able to determine the symptoms linked to sensitive skins. In particular, these symptoms are subjective signs, which are essentially dysaesthesic sensations. Dysaesthesic sensations are understood as meaning more or less painful sensations felt in a cutaneous zone, such as stinging, tingling, itching or pruritis, burning, heating, discomfort, pulling, etc.

The applicant has additionally been able to show that a sensitive skin was not an allergic skin. In fact, an allergic skin is a skin which reacts to an external agent, an allergen, which triggers an allergic reaction. It is an immunological process which only occurs when an allergen is present and which only affects sensitized subjects. According to the applicant, the essential characteristic of sensitive skin, on the contrary, is a response mechanism to external factors, which can affect any individual, even if the individuals supposedly with sensitive skin react to it more quickly than others. This mechanism is not immunological, it is non-specific.

The Applicant found that sensitive skins could be divided into two main clinical forms, irritable and/or reactive skins, and intolerant skins.

An irritable and/or reactive skin is a skin which reacts with pruritis, that is to say with itching or with stinging, to different factors such as the environment, the emotions, food, the wind, friction, razor, soap, surfactants, hard water with a high concentration of lime, variations in temperature or wool. In general, these signs are associated with a dry skin with or without scurf or a skin which exhibits erythema.

An intolerant skin is a skin which reacts with sensations of heating, pulling, tingling and/or redness, to different factors such as the environment, the emotions, food and certain cosmetic products. In general, these signs are associated with a hyperseborrhoeic or acneic skin with or without scurf and with an erythema.

“Sensitive” scalps have a more unequivocal clinical semeiology: the sensations of pruritis and/or of stinging and/or of heating are essentially triggered by local factors such as friction, soap, surfactants, hard water with a high concentration of lime, shampoos or lotions. These sensations are also sometimes triggered by factors such as the environment, the emotions and/or food. An erythema and a hyperseborrhoea of the scalp as well as a pellicular state are frequently associated with the preceding signs.

In addition, in certain anatomical regions such as the main folds (inguinal, genital, axillary, popliteal, anal, sub-mammary, elbow-fold regions) and the feet, sensitive skin is manifested by pruritic sensations and/or dysaesthesic sensations (heating, stinging) linked in particular to perspiration, to friction, to wool, to surfactants, to certain cosmetic preparations, to hard water with a high concentration of lime and/or to variations in temperature.

To determine if a skin is sensitive or not, the Applicant has likewise perfected a test. In fact, after having carried out a large number of tests with the aim of defining a sensitive skin, it found that a link existed between persons with sensitive skin and those who reacted to a topical application of capsaicin.

The capsaicin test consists in applying 0.05 ml of a cream comprising 0.075% of capsaicin on approximately 4 cm² of skin and in noting the appearance of subjective signs caused by this application, such as stinging, burning and itching. In subjects with sensitive skin, these signs appear between 3 and 20 minutes after application and are followed by the appearance of an erythema which begins at the periphery of the application zone.

Until now, capsaicin was used as a medicament, in particular to treat the pain of shingles. Capsaicin causes a release of neuropeptides, and in particular of the tachykinins which arise from nerve ends in the epidermis and the dermis. The applicant has noted that the physiopathological scheme common to all the states of sensitive skin was the aptitude to liberate, starting from sensitive cutaneous nerve fibres (fibre C), different biological mediators such as tachykinins, especially substance P, calcitonin-derived peptide (CGRP) and/or nitrogen monoxide (NO), the latter being liberated under the dependence of a constitutive NO synthase. It is additionally known that substance P liberated by sensitive epidermal nerve endings induces a degranulation of the blood cells involved in inflammation (mastocytes, monocytes, macrophages) with liberation of different mediators such as histamine, serotonin, interleukins, heparin, tumour necrosis factor of type α (TNF-α). This cascade of biochemical events results in an inflammatory reaction in which the nitrogen monoxide (liberated under the dependence of an inducible NO synthase) is likewise involved. The dysaesthec manifestations which are thus provoked are called “neurogenic”.

Nobody had established a link between nitrogen monoxide (NO) and sensitive skin. The clinical signs of sensitive skin are essentially subjective: stinging, tingling, pruritis, pulling, heating, and they are sometimes associated with erythema. These signs are due to non-specific external factors. The symptoms appear to be essentially localized to the face, to the neck and to the scalp, but can also appear all over the body.

Thus, the applicant has discovered that one of the essential characteristics of sensitive skin is linked to the liberation of nitrogen monoxide and thus that the use of inhibitor of the enzyme linked to this liberation, NO synthase, can allow a preventive and/or curative effect on sensitive skins to be obtained.

The term NO synthase in fact covers a family of enzymes which in a manner specific to the tissue assure the enzymatic catalysis of L-arginine to citrulline, a catalysis in the course of which is produced a gaseous mediator with multiple functions, nitrogen monoxide or NO. Nitrogen monoxide through its structure possesses a supplementary electron making it extremely chemically reactive. It is well known that such compounds are noxious and it is sought to limit their production as well as possible. It is for this reason that in the case of nitrogen monoxide the inhibitors of NO synthase have been widely studied.

The NO synthases exist in two forms, a constitutive form, the nomenclature grouping together neuronal NO synthase (or NOS 1) and endothelial NO synthase (or NOS 3) and the inducible form (or NOS 2) (Medecine/Sciences, 1992, 8, pp. 843-845). According to the invention, inhibitors of the constitutive form or of the inducible form are used. The tests for identifying the inhibitors of constitutive or inducible NO synthase are described especially in the U.S. Pat. No. 5,132,453.

The inhibitors of NO synthases are thus chosen among the compounds inhibiting the synthesis and/or accelerating the catabolism of NO synthase, the compounds neutralizing NO synthase or the compounds intervening by modulating the signal transmitted by NO synthase.

Thus, according to the invention the inhibitors of NO synthase are products which in situ in man allow the synthesis of nitrogen monoxide (NO) to be inhibited partially, or even totally. To treat sensitive skin, the Applicant has thus envisaged the use of inhibitors of NO synthase. It has in fact noted in a surprising manner that the incorporation of an NO synthase inhibitor in a composition intended for topical use allows irritation and/or dysaesthetic sensations and/or pruritis of the skin to be avoided.

The invention thus relates more particularly to the use of at least one NO synthase inhibitor in a cosmetic composition as active principal for treating and/or preventing sensitive skin or for the preparation of a pharmaceutical composition intended to treat sensitive skin.

Thus, the NO synthase inhibitor can be chosen among optionally modified, synthetic or natural peptides, synthetic or natural chemical molecules, antisense nucleic acids, ribozymes, anti-NO synthase antibodies.

Among these inhibitors of NO synthase, the following can be mentioned especially

N^{G}-monomethyl-L-arginine (NMMA), N^{G}-nitro-L-arginine,

N^{G}-nitro-L-arginine methyl ester, diphenyleneiodonium chloride, 7-nitroindazole,

N(5)-(1-iminoethyl)-L-ornithine, N^{G},N^{G}-dimethyl-L-arginine,

N^{G},N^{G}-dimethylarginine,

2-(4-carboxyphenyl)-4,4,5,5-tetramethylimidazoline-1-oxy 3-oxide, aminoguanidine, canavanine and ebselen.

Among the inhibitors of NO synthase, N^{G}-nitro-L-arginine methyl ester or N^{G},N^{G}-dimethyl-arginine are used preferentially.

The inhibitors of NO synthase can be used on their own or as a mixture.

The present invention also relates to the use of at least one NO synthase inhibitor in a cosmetic composition or for the preparation of a pharmaceutical composition intended to prevent and/or to combat cutaneous irritations and/or scurf and/or erythema and/or heating sensations and/or dysaesthesia and/or pruritis of the skin and/or mucous membranes.

According to the invention, the NO synthase inhibitor can be used in a quantity by weight representing from 10⁻⁶% to 10% of the total weight of the composition and preferentially in a quantity representing from 10⁻⁴% to 1% of the total weight of the composition.

The NO synthase inhibitor can be used in a composition which has to be ingested, injected or preferably applied to the skin (on any cutaneous zone of the body), the hair, the nails or the mucous membranes (buccal, jugal, gingival, genital, anal, conjunctival). According to the mode of administration, this composition can be present in any of the pharmaceutical forms normally used.

For topical application to the skin, the composition can especially have the form of an aqueous solution or oily suspension or of a dispersion of the lotion or serum type, of emulsions of liquid or semi-liquid consistency of the milky type, obtained by dispersion of a fatty phase in an aqueous phase (O/W) or conversely (W/O) or of suspensions or emulsions of soft consistency of the aqueous or anhydrous cream or gel type, or even of microcapsules or microparticles, or of vesicular dispersions of the ionic and/or non-ionic type. These compositions are prepared according to the usual methods.

They can likewise be used for the hair in the form of aqueous, alcoholic or aqueous/alcoholic solutions, or in the form of creams, gels, emulsions, or additionally in the form of aerosol compositions likewise comprising a propellant under pressure.

For injection, the composition can be present in the form of aqueous lotion, oily suspension or in the form of serum. For the eyes, it can be present in the form of drops and for ingestion, it can be present in the form of capsules, granules, syrups or tablets.

The quantities of the different constituents of the compositions according to the invention are those conventionally used in the fields considered.

These compositions are especially creams for cleansing, protection, treatment or for care of the face, hands, feet, main anatomical folds or the body, (for example day creams, night creams, make-up removal creams, foundation creams, sun protection creams), liquid foundations, make-up removal milks, body milks for protection or care, sun protection milks, lotions, gels or foams for the care of the skin, such as cleansing lotions, sun protection lotions, artificial bronzing lotions, bath compositions, deodorant compositions comprising a bactericidal agent, after-shave lotions or gels, depilatory creams, compositions against insect stings, analgesic compositions, compositions for treating certain diseases of the skin such as eczema, rosacea, psoriasis, lichen, severe pruritis.

The compositions can likewise consist of solid preparations forming soaps or cleansing blocks.

The compositions can also be formulated in the form of an aerosol composition likewise comprising a propellant under pressure.

The NO synthase inhibitor used according to the invention can also be incorporated into various compositions for hair care, and especially shampoos, curling lotions, treating lotions, hairdressing creams or gels, dye compositions (especially oxidation dyes) optionally in the form of colouring shampoos, restructuring lotions for the hair, permanent-wave compositions (especially compositions for permanent waving for the first time), holding lotions or gels, antiparasitic shampoos, etc.

The compositions can also be for bucco-dental use, for example a dentifrice paste. In this case, the compositions can contain adjuvants and additives usual for compositions for buccal use and especially surface-active agents, thickening agents, humectant agents, polishing agents such as silica, various active ingredients such as fluorides, in particular sodium fluoride, and possibly sweetening agents such as sodium saccharinate.

When the composition is an emulsion, the proportion of the fatty phase can range from 5% to 80% by weight, and preferably from 5% to 50% by weight, with respect to the total weight of the composition. The oils, the waxes, the emulsifiers and the coemulsifiers used in the composition in the form of emulsion are chosen among those conventionally used in the cosmetic field. The emulsifier and the coemulsifier are present, in the composition, in a proportion ranging from 0.3% to 30% by weight, and preferably from 0.5 to 20% by weight with respect to the total weight of the composition. The emulsion can, additionally, contain lipid vesicles.

When the composition is a solution or an oily gel, the fatty phase can represent more than 90% of the total weight of the composition.

In a known manner, the cosmetic composition can likewise contain customary adjuvants in the cosmetic field, such as hydrophilic or lipophilic gelling agents, hydrophilic or lipophilic additives, preservatives, antioxidants, solvents, perfumes, bulking agents, filters, odour absorbers or colouring materials. The quantities of these different adjuvants are those conventionally used in the cosmetic field, and are, for example, from 0.01% to 10% of the total weight of the composition. These adjuvants, according to their nature, can be introduced into the fatty phase, into the aqueous phase and/or into the lipid spheres.

As oils or waxes utilizable in the invention, it is possible to mention mineral oils (vaseline oil), vegetable oils (liquid fraction of karite butter, sunflower oil), animal oils (perhydrosqualene), synthetic oils (Purcellin oil), siliconized oils or waxes (cyclomethicone) and fluorinated oils (perfluoropolyethers), beeswax, carnauba wax or paraffin wax. It is possible to add to these oils fatty alcohols and fatty acids (stearic acid).

As emulsifiers utilizable in the invention, it is possible to mention, for example, glycerol stearate, polysorbate 60 and the mixture of PEG-6/PEG-32/glycol stearate sold under the name Tefose® 63 by Gattefosse.

As solvents utilizable in the invention, it is possible to mention lower alcohols, especially ethanol and isopropanol, and propylene glycol.

As hydrophilic gelling agents utilizable in the invention, it is possible to mention carboxyvinyl polymers (carbomer), acrylic copolymers such as acrylate/alkylacrylate copolymers, polyacrylamides, polysaccharides such as hydroxypropylcellulose, natural rubbers and clays, and, as lipophilic gelling agents, it is possible to mention modified clays such as bentonites, metal salts of fatty acids such as aluminium stearates and hydrophobic silica, ethyl-cellulose and polyethylene.

The composition can contain other hydrophilic agents such as proteins or protein hydrolysates, amino acids, polyols, urea, allantoin, sugars and sugar derivatives, water-soluble vitamins, vegetable extracts and hydroxyacids.

As lipophilic active compounds, it is possible to use retinol (vitamin A) and its derivatives, tocopherol (vitamin E) and its derivatives, essential fatty acids, ceramides, essential oils, salicylic acid and its derivatives.

According to the invention, it is possible, among other things, to associate at least one NO synthase inhibitor with other active agents especially intended for the prevention and/or for the treatment of cutaneous disorders.

Among these active agents, it is possible to mention by way of example:

agents modulating cutaneous differentiation and/or proliferation and/or pigmentation such as retinoic acid and its isomers, retinol and its esters, vitamin D and its derivatives, oestrogens such as oestradiol, kojic acid or hydroquinone;

antibacterial agents such as clindamycin phosphate, erythromycin or antibiotics of the tetracycline class;

antiparasitics, in particular metronidazole, crotamiton or pyrethroids;

antifungals, in particular compounds belonging to the imidazoles class such as econazole, ketoconazole or miconazole or their salts, polyene compounds, such as amphotericin B, compounds of the allylamine family, such as terbinafine, or additionally octopirox;

antiviral agents such as acyclovir;

steroidal anti-inflammatory agents, such as hydrocortisone, betamethasone valerate or clobetasol propionate, or non-steroidal anti-inflammatory agents such as ibuprofen and its salts, diclofenac and its salts, acetylsalicylic acid, acetaminophen or glycyrrhetic acid;

anaesthetic agents such as lidocaine hydrochloride and its derivatives;

antipruritic agents such as thenaldine, trimeprazine or cyproheptadine;

keratolytic agents such as alpha- and beta-hydroxycarboxylic or beta-ketocarboxylic acids, their salts, amides or esters and more particularly hydroxy acids such as glycolic acid, lactic acid, salicylic acid, citric acid and, generally speaking, fruit acids, and n-octanoyl-5-salicylic acid;

anti-free radical agents, such as alpha-tocopherol or its esters, superoxide dismutases, certain metal chelating agents or ascorbic acid and its esters;

antiseborrhoeic agents such as progesterone;

antidandruff agents such as octopirox or zinc pyrithione;

antiacne agents such as retinoic acid or benzoyl peroxide.

Thus, according to a particular mode, the invention relates to the use of at least one NO synthase inhibitor in a composition comprising at least one agent chosen among the antibacterial, antiparasitic, antifungal, antiviral, anti-inflammatory, antipruritic, anaesthetic, keratolytic, anti-free radical, anti-seborrhoeic, antidandruff or antiacne agents and/or agents modulating cutaneous differentiation and/or proliferation and/or pigmentation.

The following examples of compositions illustrate the invention without limiting it in any way. The proportions indicated are percentages by weight. These compositions were obtained by simple mixing of the different components.

| | |
|---|---|
| Composition 1: | |
| Make-up removal lotion for the face | |
| N^{G}-monomethyl-L-arginine (NMMA) | 10⁻⁵% |
| Antioxidant | 0.05% |
| Isopropanol | 40.00% |
| Preservative | 0.30% |
| Water | qs 100% |
| Composition 2: | |
| Gel for care of the face | |
| 7-nitroindazole | 10⁻⁴% |
| Hydroxypropylcellulose (Klucel H | 1.00% |
| sold by Hercules) | |
| Antioxidant | 0.05% |
| Isopropanol | 40.00% |
| Preservative | 0.30% |
| Water | qs 100% |
| Composition 3: | |
| Face-care cream (oil-in-water emulsion) | |
| N^{G}, N^{G}-dimethyl-L-arginine | 10⁻²% |
| Glycerol stearate | 2.00% |
| Polysorbate 60 (Tween 60 sold by ICI) | 1.00% |
| Stearic acid | 1.40% |
| Triethanolamine | 0.70% |
| Carbomer | 0.40% |
| Liquid fraction of karite butter | 12.00% |
| Perhydrosqualene | 12.00% |
| Antioxidant | 0.05% |
| Perfume | 0.50% |
| Preservative | 0.30% |
| Water | qs 100% |
| Composition 4: | |
| Shampoo | |
| N^{G}, N^{G}-dimethyl-L-arginine | 10⁻⁵% |
| Na lauryl ether sulphate (2.2 OE) | 12.00% |
| Hydroxypropylcellulose (Klucel H | 1.00% |
| sold by Hercules) | |
| Perfume | 0.50% |
| Preservative | 0.30% |
| Water | qs 100% |
| Composition 5: | |
| Analgesic gel | |
| N^{G}-monomethyl-L-arginine (NMMA) | 1.00% |
| Hydroxypropylcellulose (Klucel H | 1.00% |
| sold by Hercules) | |
| Antioxidant | 0.05% |
| Lidocaine hydrochloride | 2.00% |
| Isopropanol | 40.00% |
| Preservative | 0.30% |
| Water | qs 100% |
| Composition 6: | |
| Sunburn-care cream (oil-in-water emulsion) | |
| 7-nitroindazole | 0.50% |
| Glycerol stearate | 2.00% |
| Polysorbate 60 (Tween 60 sold by ICI) | 1.00% |
| Stearic acid | 1.40% |
| Glycyrrhetic acid | 2.00% |
| Triethanolamine | 0.70% |
| Carbomer | 0.40% |
| Liquid fraction of karite butter | 12.00% |
| Sunflower oil | 10.00% |
| Antioxidant | 0.05% |
| Perfume | 0.50% |
| Preservative | 0.30% |
| Water | qs 100% |

1. Use of at least one NO synthase inhibitor in a cosmetic composition as active principle for treating and/or preventing sensitive skins.

2. Use of at least one NO synthase inhibitor for the preparation of a pharmaceutical composition intended for the treatment of sensitive skins.

3. Use according to one of the preceding claims, characterized in that the cosmetic or pharmaceutical composition is intended to prevent and/or combat cutaneous irritations and/or scurf and/or erythema and/or dysaesthec sensations and/or sensations of heating and/or pruritus of the skin and/or the mucous membranes.

4. Use according to any one of claims 1 to 3, characterized in that the NO synthase inhibitor is used in a quantity representing from 10⁻⁶% to 10% of the total weight of the composition.

5. Use according to claim 4, characterized in that the NO synthase inhibitor is used in a quantity representing from 10⁻⁴% to 1% of the total weight of the composition.

6. Use according to any one of the preceding claims, characterized in that the NO synthase inhibitor is chosen amongst N^{G}-monomethyl-L-arginine, N^{G}-nitro-L-arginine methyl ester, N^{G}-nitro-L-arginine, diphenyleneiodonium chloride, 7-nitroindazole, N(5)-(1-iminoethyl)-L-ornithine, 2-(4-carboxyphenyl)-4,4,5,5-tetramethylimidazoline-1-oxy 3-oxide, N^{G},N^{G}-dimethyl-L-arginine, N^{G},N^{G}-dimethylarginine, aminoguanidine, canavanine and ebselen.

7. Use according to claim 6, characterized in that the NO synthase inhibitor is N^{G}-nitro-L-arginine methyl ester or N^{G},N^{G}-dimethylarginine.
